(19) United States
(12) Patent Application Publication
(10) Pub. No.: US 2020/0347446 A1
(43) Pub. Date: Nov. 5, 2020

(54) METHODS AND SYSTEMS FOR PROCESSING NUCLEIC ACID MOLECULES

(71) Applicant: 10X GENOMICS, INC., Pleasanton, CA (US)

(72) Inventors: Zahra Kamila Belhocine, Fremont, CA (US); Francesca Meschi, Menlo Park, CA (US); Andrew D. Price, Hayward, CA (US); Yifeng Yin, Elk Grove, CA (US)

(21) Appl. No.: 16/877,964

(22) Filed: May 19, 2020

Related U.S. Application Data
(63) Continuation of application No. PCT/US2018/064103, filed on Dec. 5, 2018.
(60) Provisional application No. 62/595,424, filed on Dec. 6, 2017.

(51) Int. Cl. C12Q 1/6855; C12Q 1/6806

(52) U.S. Cl. CPC C12Q 1/6855; C12Q 1/6806

(57) ABSTRACT

Provided herein are methods and systems for processing nucleic acid molecules in partitions. The nucleic acid molecules may be fragmented using restriction enzymes and barcoded in the partitions.

### CROSS-REFERENCE

This application is a continuation of International Application No. PCT/US2018/064103, filed Dec. 5, 2018, which claims the benefit of U.S. Provisional Patent Application No. 62/595,424, filed Dec. 6, 2017, which applications are entirely incorporated herein by reference.

### BACKGROUND

Samples may be processed for various purposes, such as identification of a type of sample of moiety within the sample. The sample may be a biological sample. The biological samples may be processed for various purposes, such as detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

### SUMMARY

An aspect of the present disclosure provides methods for processing a plurality of nucleic acid molecules, comprising: (a) providing a plurality of partitions comprising the plurality of nucleic acid molecules, wherein a given partition of the plurality of partitions comprises (i) a given nucleic acid molecule of the plurality of nucleic acid molecules, wherein the given nucleic acid molecule comprises at least one nucleotide analog, and (ii) a modification dependent restriction enzyme; and (b) in the given partition, using the modification dependent restriction enzyme to subject the given nucleic acid molecule to fragmentation at a location at or in proximity to the at least one nucleotide analog to yield a plurality of nucleic acid fragments; and (c) using an oligonucleotide barcode molecule comprising a barcode sequence to barcode a given nucleic acid fragment of the plurality of nucleic acid fragments, to yield a barcoded fragment comprising the barcode sequence.

In some embodiments, the method further comprises, prior to (a), subjecting a nucleic acid molecule to nucleic acid amplification to yield the given nucleic acid molecule comprising the at least one nucleotide analog. In some cases, the amplification is performed at temperatures between 25° C. and 35° C. In some cases, the amplification is performed using a polymerizing enzyme. In some cases, the polymerizing enzyme is a high fidelity polymerizing enzyme. In some cases, the high fidelity polymerizing enzyme is phi29 or a functional derivative thereof. In some cases, the amplification is performed in the given partition. In some cases, the amplification is performed prior to providing the plurality of nucleic acid molecules in the plurality of partitions.

In some embodiments, the nucleic acid molecule is from a single cell or cell bead. In some cases, the single cell or cell bead is in the given partition.

In some embodiments, the nucleotide analog is a methylated nucleotide analog.

In some embodiments, the modification dependent restriction enzyme is a methylation dependent restriction enzyme. In some cases, the modification dependent restriction enzyme is a blunt cutter that yields a blunt ended fragmented nucleic acid molecule. In some cases, the modification dependent restriction enzyme yields a cohesive ended fragmented nucleic acid molecule. In some cases, the modification dependent restriction enzyme fragments the nucleic acid molecule at the nucleotide analog. In some cases, the modification dependent restriction enzyme fragments the nucleic acid molecule at a position proximal to the nucleotide analog. In some cases, the modification dependent restriction enzyme fragments the nucleic acid molecule at a position up to 2 nucleotide bases from the nucleotide analog. In some cases, the modification dependent restriction enzyme fragments the nucleic acid molecule at a position up to 5 nucleotide bases from the nucleotide analog. In some cases, the modification dependent restriction enzyme fragments the nucleic acid molecule at a position up to 10 nucleotide bases from the nucleotide analog.

In some embodiments, fragmentation of the nucleic acid molecule by the restriction enzyme may be performed at temperatures between 30° C. and 40° C. In some embodiments, the fragmented nucleic acid comprises fragments in a size range from about 100 to 1000 nucleotide bases.

In some embodiments, the given partition comprises the oligonucleotide barcode molecule comprising the barcode sequence.

In some embodiments, barcoding of the fragmented nucleic acid is performed in the given partition. In some embodiments, the barcode is coupled to a bead. In some cases, the bead is a gel bead.

In some embodiments, the oligonucleotide barcode molecule is from a plurality of oligonucleotide barcode molecules comprising first barcode sequences and second barcode sequences, wherein the first barcode sequences are the same across the plurality of oligonucleotide barcode molecules, and wherein the second barcode sequences are different across the plurality of oligonucleotide barcode molecules.

In some embodiments, the method further comprises removing the barcoded fragment or derivative thereof from the given partition.

In some embodiments, the given partition is a droplet as part of an emulsion comprising a plurality of droplets, and wherein the removing comprises disrupting the emulsion to release the barcoded fragment or derivative thereof from the given partition.

In some embodiments, the method further comprises subjecting the barcoded fragment or derivative thereof to nucleic acid amplification upon removing the barcoded fragment or derivative thereof from the given partition. In some cases, the nucleic acid amplification is polymerase chain reaction (PCR). In some cases, the PCR is isothermal PCR. In some cases, the PCR comprises thermal cycling.

In some embodiments, the method further comprises performing one or more reactions on the barcoded fragment. In some cases, the one or more reactions are performed in the given partition. In some cases, the one or more reactions are performed external to the given partition. In some cases, the one or more reactions comprise performing nucleic acid amplification. In some cases, the one or more reactions comprise coupling a flow cell sequence to the barcoded fragment or derivative thereof, which flow cell sequence permits attachment of the barcoded fragment or derivative thereof to a flow cell of a sequencer.

In some embodiments, the method further comprises subjecting the barcoded fragment or derivative thereof to nucleic acid sequencing to identify a sequence of at least a portion of the given nucleic acid fragment and at least a portion of the barcode sequence.

In some embodiments, the plurality of partitions is a plurality of droplets. In some cases, the plurality of partitions is a plurality of wells.

In some embodiments, the given nucleic acid fragment is barcoded upon ligating the oligonucleotide barcode molecule to the given nucleic acid fragment. In some cases, the oligonucleotide barcode molecule is ligated using ligase. In some cases, the ligase is T4 DNA ligase or a functional derivative thereof. In some cases, the ligation is performed at a temperature between about 16° C. and 30° C. In some cases, the given nucleic acid fragment is barcoded using the oligonucleotide barcode molecule to amplify the given nucleic acid fragment.

In another aspect, the present disclosure provides a library of partitions, comprising a plurality of partitions comprising a plurality of nucleic acid molecules, wherein a given partition of the plurality of partitions comprises (i) a given nucleic acid molecule of the plurality of nucleic acid molecules, wherein the given nucleic acid molecule comprises at least one nucleotide analog, (ii) a modification dependent restriction enzyme, wherein the modification dependent restriction enzyme is configured to subject the given nucleic acid molecule to fragmentation at a location at or in proximity to the at least one nucleotide analog to yield a plurality of nucleic acid fragments, and (iii) an oligonucleotide barcode molecule comprising a barcode sequence, wherein the oligonucleotide barcode molecule is configured to barcode a given nucleic acid fragment of the plurality of nucleic acid fragments to yield a barcoded fragment comprising the barcode sequence.

In some embodiments, the given partition comprises a polymerizing enzyme. In some cases, the polymerizing enzyme is a high fidelity polymerizing enzyme. In some cases, the high fidelity polymerizing enzyme is phi29 or a functional derivative thereof.

In some embodiments, the given partition comprises a single cell or cell bead.

In some embodiments, the nucleotide analog is a methylated nucleotide analog.

In some embodiments, the modification dependent restriction enzyme is a methylation dependent restriction enzyme. In some cases, the modification dependent restriction enzyme is a blunt cutter that yields a blunt ended fragmented nucleic acid molecule. In some cases, the modification dependent restriction enzyme is configured to yield a cohesive ended fragmented nucleic acid molecule. In some cases, the modification dependent restriction enzyme is configured to fragment the nucleic acid molecule at the nucleotide analog. In some cases, the modification dependent restriction enzyme is configured to fragment the nucleic acid molecule at a position proximal to the nucleotide analog. In some cases, the modification dependent restriction enzyme is configured to fragment the nucleic acid molecule at a position up to 2 nucleotide bases from the nucleotide analog. In some cases, the modification dependent restriction enzyme is configured to fragment the nucleic acid molecule at a position up to 5 nucleotide bases from the nucleotide analog. In some cases, the modification dependent restriction enzyme is configured to fragment the nucleic acid molecule at a position up to 10 nucleotide bases from the nucleotide analog.

In some embodiments, the fragmentation of the nucleic acid molecule by the restriction enzyme may be performed at temperatures between 30° C. and 40° C. In some embodiments, the fragmented nucleic acid comprises fragments in a size range from about 100 to 1000 nucleotide bases.

In some embodiments, the given partition comprises the oligonucleotide barcode molecule comprising the barcode sequence. In some cases, the barcode is coupled to a bead. In some cases, the bead is a gel bead.

In some embodiments, the oligonucleotide barcode molecule is from a plurality of oligonucleotide barcode molecules comprising first barcode sequences and second barcode sequences, wherein the first barcode sequences are the same across the plurality of oligonucleotide barcode molecules, and wherein the second barcode sequences are different across the plurality of oligonucleotide barcode molecules.

In some embodiments, the plurality of partitions is a plurality of droplets. In some cases, the plurality of partitions is a plurality of wells.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7 shows an example of a method for processing nucleic acid molecules.

FIG. 8 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. The subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient.

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases (or nucleic acid bases) in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, generally refers to a biological sample of a subject. The biological sample may comprise any number of macromolecules, for example, cellular macromolecules. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may small RNA that are less than 200 nucleic acid bases in length, or large RNA that are greater than 200 nucleic acid bases in length. Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The terms “template nucleic acid”, “target nucleic acid”, “nucleic acid molecule,” “nucleic acid sequence,” “nucleic acid fragment,” “oligonucleotide” and “polynucleotide,” as used herein, generally refer to a polynucleotide that may have various lengths, such as either deoxy ribonucleotides or deoxyribonucleic acid (DNA) or ribonucleotides or ribonucleic acid (RNA), or analogs thereof. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence and isolated RNA of any sequence.

The term “partition,” as used herein, generally, refers to a space or volume that may be suitable to contain one or more species or conduct one or more reactions. The partition may isolate space or volume from another space or volume. The partition may be a droplet or well, for example. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase.

As used herein, the term “nucleotide analog” generally refers to a compound that is structurally analogous to a naturally occurring nucleotide. Nucleotide analogs may be non-naturally occurring or non-canonical. Examples of nucleotide analogs include, but are not limited to, locked nucleic acid bases, diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, 5-hydroxymethylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, 2,6-diaminopurine, phosphoroselenoate nucleic acids and the like.

The term “polymerase,” as used herein, generally refers to any enzyme capable of catalyzing a polymerization reaction. Polymerases may be used extend primers with the incorporation of nucleotides or nucleotide analogs. Examples of polymerases include, without limitation, a nucleic acid polymerase. The polymerase can be naturally occurring or synthesized. In some cases, a polymerase has relatively high processivity. An example polymerase is a (29 polymerase or a derivative thereof. A polymerase can be a polymerization enzyme. In some cases, a transcriptase is used. Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof.

As used herein, the term “restriction enzyme” generally refers to an enzyme (e.g., restriction endonuclease, etc.) that cuts DNA at sites within the DNA molecule. Restriction enzymes used herein may be specific to a recognition site and may fragment the nucleic acid molecules at a location at or in proximity to the restriction site. Restriction enzymes used may be naturally occurring enzymes or they may be modified. The restriction enzymes may be modified to detect a specific restriction site or a nucleotide analog. Alternatively or in addition, the restriction enzyme used may naturally be specific for a nucleotide analog and may fragment the nucleic acid molecules at a location or in proximity to the restriction site or nucleotide analog. In some cases, the restriction enzymes used are modification dependent restriction enzymes. In such examples, the restriction enzymes may specifically fragment the nucleic acid molecules only in the presence of a nucleotide analog and may not be able to fragment nucleic acid molecules in the absence of a nucleotide analog.

Non-limiting examples of restriction enzymes include: AatII, AbaSI, Acc65I, AccI, AcdI, AciI, AclI, AcuI, AfeI, AflII, AflIII, AgeI, AhdI, AleI, AluI, AlwI, AlwNI, ApaI, ApaLI, ApeKI, ApoI, SoxI, AscI, AseI, AsiSI, AspBHI, AvaI, AvaII, AvrII, BaeGI, BaeI, BamHI, BanI, BanII, BbeI, BbsI, BbvCI, BbvI, BccI, BceAI, BcgI, BciVI, BclI, BcoDI, BfaI, BfuAI, BglI, BgII, BlpI, BisI, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssS_I, BstAPI, BstBI, BstEII, BstNI, BstUI, BstXI, BstYI, Bsu36I, BtgI, BtgZI, BtsCI, BtsIMutI, Bts_I, Cac8I, ClaI, CspCI, CviAII, CviKIl, CviQI, DdeI, DpnI, DpnII, DraI, DrdI, EaeI, EagI, Earl, EciI, Eco53kI, EcoK, EcoNI, EcoO109I, EcoP15I, EcoRI, EcoRV, Esp3I, FatI, FauI, Fnu4HI, FokI, FseI, FspEI, FspI, Glal, GluI, HaeII, HaeIII, HgaI, HhaI, HincII, HindIII, Hinfi, HinPlI, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, KroI, LpnI, LpnPI, MalI, MboI, MboII, McrA, McrBC, MfeI, MluCI, MluI, MlyI, MmeI, MnlI, MrrIA, MscI, MseI, MslI, MspAlI, MspI, MspJI, MteI, MwoI, NaeI, Narl, Nb.BbvCI, Nb.BsmI, Nb.BsrDI, Nb.BssSI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PsI, PflFI, PflMI, PkrI, PI-PspI, PI-SceI, PleI, PluTI, PmeI, PmlI, PpuMI, PshAI, PsiI, PspGI, PspOMI, PspXI, PstI, PvuI, PvuII, RlaI, RsaI, RsrII, SacI, SacII, SalI, SapI, Sau3AI, Sau96I, SauNewI, SauUSI, SbfI, ScoA3, ScrFI, SepR, SexAl, SfaNI, SfcI, SfiI, SfoI, SgeI, SgrAI, SmaI, SmlI, SnaBI, SpeI, SphI, Srfl, SspI, StuI, StyD4I, StyI, SwaI, Taq_I, TfiI, TseI, Tsp45I, TspMI, TspRI, Tth111I, XbaI, XcmI, XhoI, XmaI, XmnI, ZmoMrr. Restriction enzymes used may be any suitable commercially available restriction enzyme. The restriction enzymes used may create a single stranded overhang upon fragmenting the nucleic acid molecules. For example, when DNA is digested with the restriction enzyme, the resulting double stranded fragments may be flanked at either end by a single stranded overhang, which may act as a cohesive end and be used to ligate a barcode. Alternatively, digestion with the restriction enzymes may lead to a double stranded fragment with a blunt end. A restriction enzyme as used herein may be modified to fragment nucleic acid molecules and leave either blunt ends and/or single stranded overhangs.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase. Moreover, amplification of a nucleic acid may linear, exponential, or a combination thereof. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification (MDA). In cases where DNA is amplified, various DNA amplification methods may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). Amplification may be used to incorporate nucleotides and/or nucleotide analogs in to a growing chain of nucleic acids. PCR may be employed with thermal cycling or isothermally (i.e., isothermal PCR).

As used herein, the term “ligase” generally refers to an enzyme that is capable of covalently linking the 3′ hydroxyl group of a nucleotide to the 5′ phosphate group of a second nucleotide. Examples of ligases include E. coli DNA ligase, T4 DNA ligase, etc. As used herein, “ligating” refers to covalently attaching two nucleic acid molecules to form a single nucleic acid molecule. Ligases may be used to attach a barcode to a nucleic acid molecule or a fragment thereof.

The present disclosure provides methods for processing nucleic acid molecules by incorporating nucleotide bases that are complementary to a sequence of the nucleic acid molecules. Such incorporation may be performed using enzymes, such as a polymerase. In some cases, a nucleotide analog (or multiple nucleotide analogs) may be incorporated by the polymerase or functional variant into a growing chain of the nucleic acid molecule.

The nucleic acid molecule may be provided in a partition and the nucleotide analog may be incorporated in the nucleic acid molecule in the partition, or the nucleic acid molecule may be partitioned after the incorporation of the nucleotide analogs.

The nucleotide analog may be used to generate a recognition site specific to a restriction enzyme. The restriction enzyme may, for example, be a restriction endonuclease. The restriction enzyme may be present in the same partition as the nucleic acid molecule or may be provided from a separate partition. The restriction endonuclease may fragment the nucleic acid molecule at the recognition site. After fragmentation, at least some of the fragments of the nucleic acid molecule may be attached to barcodes. The attachment of the barcodes to the fragments may be performed using a ligase enzyme. The barcodes and the ligase enzyme may be present in the initial partition with the nucleic acid molecule or may be provided from a separate partition. Upon the attachment of the barcodes, the fragmented nucleic acid molecules may be further processed, for example, by nucleic acid amplification or addition of flow cell sequences. The fragmented nucleic acid molecules or derivatives thereof may be sequenced.

### Methods for Processing Nucleic Acid Molecules

In an aspect, the systems and methods described herein provide for the compartmentalization, depositing, or partitioning of macromolecular constituent contents of individual biological particles into discrete compartments or partitions (referred to interchangeably herein as partitions), where each partition maintains separation of its own contents from the contents of other partitions. The partition can be a droplet in an emulsion. A partition may comprise one or more other partitions.

A partition of the present disclosure may comprise biological particles and/or macromolecular constituents thereof. A partition may comprise one or more gel beads. A partition may comprise one or more cell beads. A partition may include a single gel bead, a single cell bead, or both a single cell bead and single gel bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described further below. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can comprise droplets of aqueous fluid within a non-aqueous continuous phase (e.g., oil phase). The partitions can comprise droplets of a first phase within a second phase, wherein the first and second phases are immiscible. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual biological particles to discrete partitions may in one non-limiting example be accomplished by introducing a flowing stream of biological particles in an aqueous fluid into a flowing stream of a non-aqueous fluid, such that droplets are generated at the junction of the two streams. By providing the aqueous stream at a certain concentration and/or flow rate of biological particles, the occupancy of the resulting partitions (e.g., number of biological particles per partition) can be controlled. Where single biological particle partitions are used, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, cell or cellular material). In some embodiments, the relative flow rates of the fluids can be selected such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

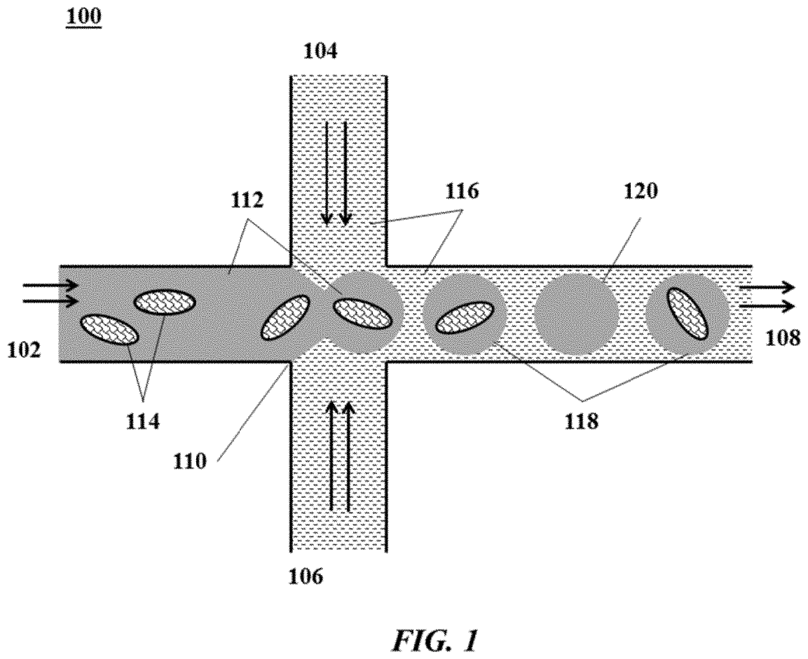

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying biological particles, cell beads, and/or gel beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

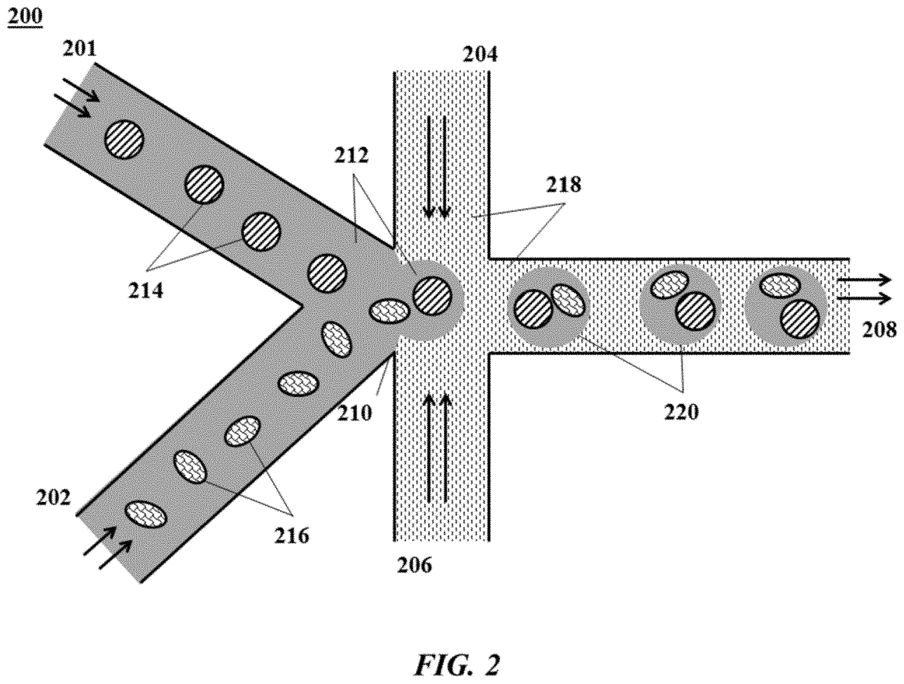

As will be appreciated, the above-described occupancy rates are also applicable to partitions that include both biological particles and additional reagents, including, but not limited to, microcapsules carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca² ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholiniumchloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles (e.g., a cell) or macromolecular constituents (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli. In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

A partition may comprise one or more unique identifiers, such as barcodes. Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Beads, biological particles and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle 216. A discrete droplet that is generated may include a barcode or other reagent carrying bead 214. A discrete droplet generated may include both an individual biological particle and a barcode carrying bead, such as droplets 220. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 1 micrometers (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds or thioether bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or a one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (μL), 800 μL, 700 μL, 600 μL, 500 μL, 400 μL, 300 μL, 200 μL, 100 μL, 50 μL, 20 μL, 10 μL, 1 μL, 500 nanoliters (nL), 100 nL, 50 nL, or less.

For example, in the case of droplet based partitions, the droplets may have overall volumes that are less than about 1000 μL, 900 μL, 800 μL, 700 μL, 600 μL, 500 μL, 400 μL, 300 μL, 200 μL, 100 μL, 50 μL, 20 μL, 10 μL, 1 μL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

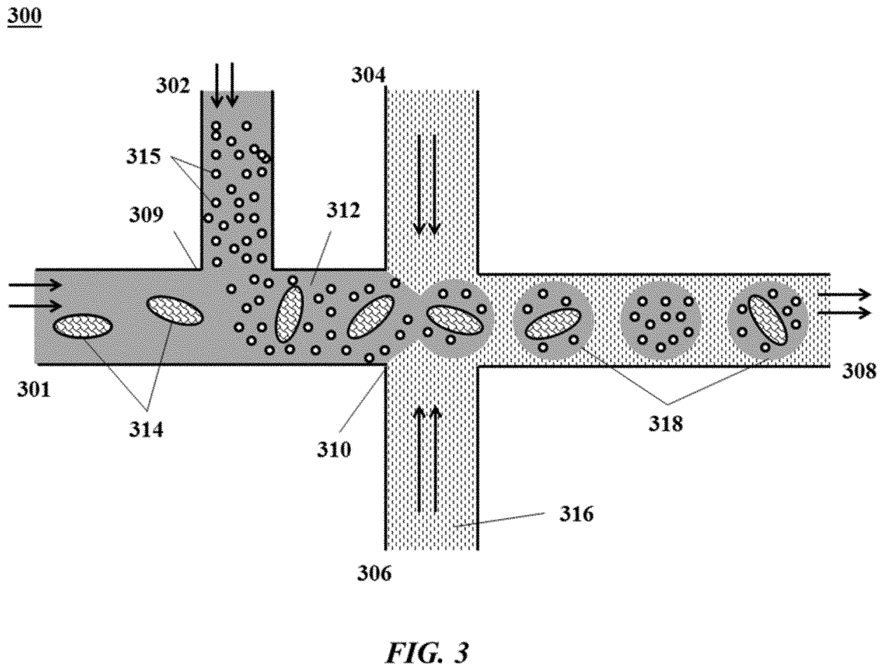

FIG. 3 shows an example of a microfluidic channel structure 300 for co-partitioning biological particles and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particles's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

In addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particle, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

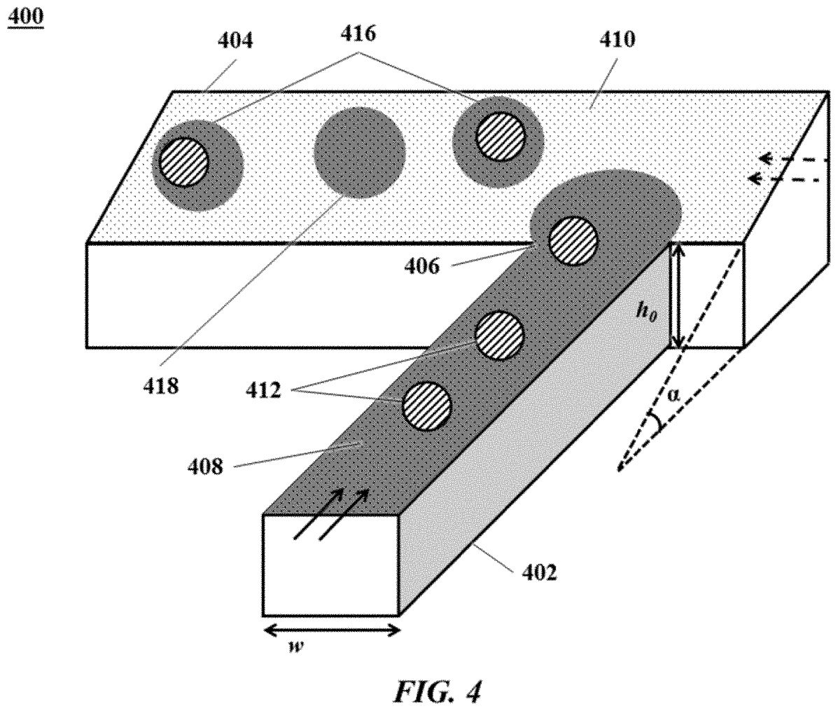

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the juncture 406 where the aqueous fluid 408 and the second fluid 410 meet, droplets can form based on factors such as the hydrodynamic forces at the juncture 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the juncture 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the juncture 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the juncture 406 may have certain geometric features that at least partly determine the sizes of the droplets formed by the channel structure 400. The channel segment 402 can have a height, h₀ and width, w, at or near the juncture 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the juncture 406 can be inclined at an expansion angle, α. The expansion angle, α, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3° 4° 5° 6°, 7° 8°, 9° 10°, 15°, 20°, 25°, 30°, 35° 40°, 45° 50°, 55° 60°, 65°, 70°, 75° 80°, 85°, or higher. In some instances, the expansion angle can be at most about 89°, 88°, 87°, 86°, 85°, 84°, 83°, 82°, 81°, 80°, 75° 70°, 65°, 60°, 55° 50°, 45° 40°, 35° 30°, 25°, 20°, 15°, 10°, 9° 8°, 7° 6°, 5° 4°, 3° 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μl/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

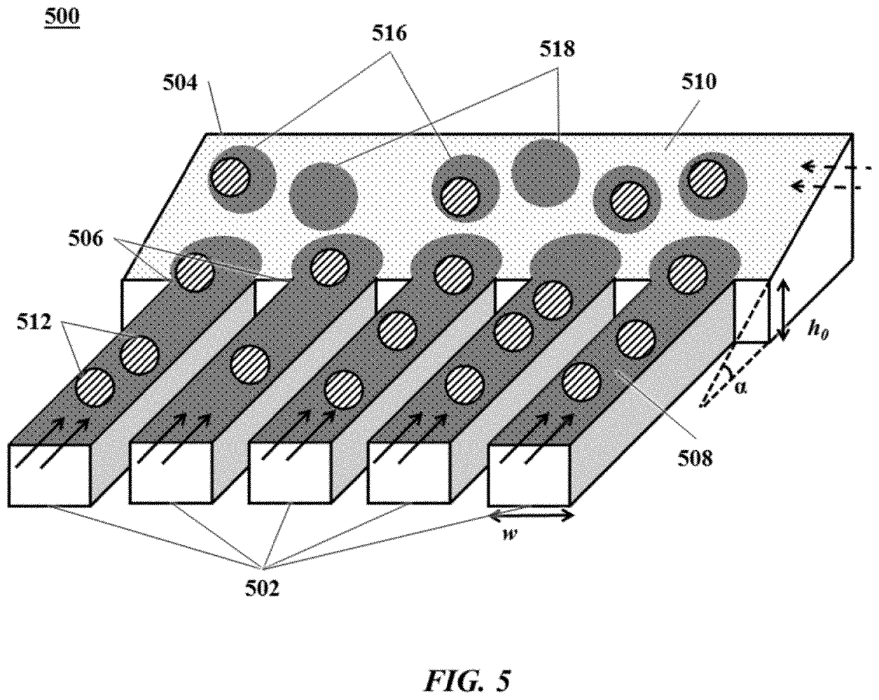

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctures. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the juncture where the aqueous fluid 508 and the second fluid 510 meet, droplets can form based on factors such as the hydrodynamic forces at the juncture, flow rates of the two fluids 508, 510, fluid properties, and certain geometric parameters (e.g., w, h₀, α, etc.) of the channel structure 500, as described elsewhere herein. A plurality of droplets can be collected in the reservoir 504 by continuously injecting the aqueous fluid 508 from the plurality of channel segments 502 through the plurality of junctures 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

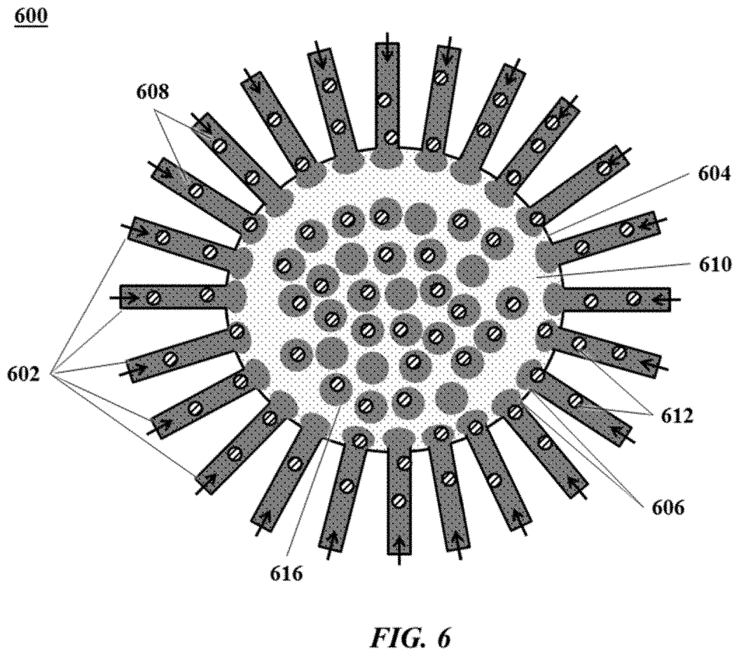

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 2 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctures. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the juncture where the aqueous fluid 608 and the second fluid 610 meet, droplets can form based on factors such as the hydrodynamic forces at the juncture, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctures 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, α (not shown in FIG. 6) at or near each channel juncture. Each channel segment of the plurality of channel segments 602 may have a width, w, and a height, h₀, at or near the channel juncture. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

In another aspect, provided herein are methods for processing a plurality of nucleic acid molecules. The methods may comprise the use of a plurality of partitions. The partitions may comprise the plurality of nucleic acid molecules. A given partition of the plurality of partitions may comprise a given nucleic acid molecule of the plurality of nucleic acid molecules. The given nucleic acid molecule may comprise at least one nucleotide analog. The given partition may comprise a modification dependent restriction enzyme. In the given partition, the modification dependent restriction enzyme may be used to subject the given nucleic acid molecule to fragmentation. The fragmentation of the nucleic acid molecule may be performed at a location at or in proximity to the at least one nucleotide analog to yield a plurality of nucleic acid fragments. Upon or during the fragmentation of the nucleic acid molecule, an oligonucleotide barcode molecule comprising a barcode sequence may be used to barcode a given nucleic acid fragment of the plurality of nucleic acid fragments and may yield a barcoded fragment comprising the barcode sequence.

In another aspect, provided herein is a library of partitions. The library of partitions may comprise a plurality of partitions. The plurality of partitions may comprise a plurality of nucleic acid molecules. A given partition of the plurality of partitions may comprise a given nucleic acid molecule of the plurality of nucleic acid molecules. The given nucleic acid molecule may comprise at least one nucleotide analog. In addition to the nucleic acid molecule, the given partition may comprise a modification dependent restriction enzyme. The modification dependent restriction enzyme may be configured to subject the given nucleic acid molecule to fragmentation. The fragmentation may be performed at a location at or in proximity to the at least one nucleotide analog to yield a plurality of nucleic acid fragments. The given partition may also comprise an oligonucleotide barcode molecule comprising a barcode sequence. The oligonucleotide barcode molecule may be configured to barcode a given nucleic acid fragment of the plurality of nucleic acid fragments to yield a barcoded fragment comprising the barcode sequence.

In another aspect, provided herein are methods for processing a plurality of nucleic acid molecules. The methods may comprise the use of a plurality of partitions. The partitions may comprise the plurality of nucleic acid molecules. A given partition of the plurality of partitions may comprise a given nucleic acid molecule of the plurality of nucleic acid molecules. The given partition may comprise at least one restriction enzyme. In the given partition, the restriction enzyme may be used to subject the given nucleic acid molecule to fragmentation to yield a plurality of nucleic acid fragments. Upon or during the fragmentation of the given nucleic acid molecule, an oligonucleotide barcode molecule comprising a barcode sequence may be used to barcode a given nucleic acid fragment of the plurality of nucleic acid fragments and may yield a barcoded fragment comprising the barcode sequence.

Prior to providing a nucleic acid molecule that may comprise a nucleotide analog, the nucleic acid molecule may be subjected nucleic acid amplification (e.g., PCR, MDA). The amplification may be performed in the presence of a nucleotide analog to yield the given nucleic acid molecule comprising at least one nucleotide analog.

The nucleic acid molecule may be from a cell. In some cases, the cell may be in the given partition. The cell may be lysed in the partition and amplified to incorporate a nucleotide analog within the partition.

The amplification reaction mixture may comprise nucleotide analogs. In some cases, the amplification reaction mixture comprises a mixture of nucleotides, e.g., dinucleotide triphosphates (dNTPs) and nucleotide analogs. For instance, the nucleotides may be 1% nucleotide analogs and 99% unmodified nucleotides (dNTPs). In some examples, the mixture comprises 99.5% dNTPs and 0.5% nucleotide analogs, 99% dNTPs and 1% nucleotide analogs, 98% dNTPs and 2% nucleotide analogs, 97% dNTPs and 3% nucleotide analogs, 95% dNTPs and 5% nucleotide analogs, 92% dNTPs and 8% nucleotide analogs, 90% dNTPs and 10% nucleotide analogs, 87% dNTPs and 13% nucleotide analogs or 85% dNTPs and 15% nucleotide analogs. In some cases, more than one type of nucleotide analogs may be present in the reaction mixture, such as the nucleotide analogs presented elsewhere herein.

In some examples, the mixture of unmodified nucleotides and nucleotide analogs comprises at least 0.01% nucleotide analogs, at least 0.02% nucleotide analogs, at least 0.05% nucleotide analogs, at least 1% nucleotide analogs, at least 2% nucleotide analogs, at least 3% nucleotide analogs, at least 5% nucleotide analogs, at least 7% nucleotide analogs, at least 10% nucleotide analogs, at least 13% nucleotide analogs, or at least 15% nucleotide analogs. A remainder of the mixture may comprise unmodified nucleotides (e.g., dNTPs). The nucleotide analogs may be modified nucleotide analogs.

In some examples, the mixture of unmodified nucleotides and nucleotide analogs comprises at most 20% nucleotide analogs, at most 15% nucleotide analogs, at most 13% nucleotide analogs, at most 10% nucleotide analogs, at most 7% nucleotide analogs, at most 5% nucleotide analogs, at most 3% nucleotide analogs, at most 2% nucleotide analogs, at most 1% nucleotide analogs, at most 0.05% nucleotide analogs, or at most 0.02% nucleotide analogs. A remainder of the mixture may comprise unmodified nucleotides (e.g., dNTPs). The nucleotide analogs may be modified nucleotide analogs. The nucleotide analogs may be methylated nucleotide analogs. In some examples, the amplification reaction may be performed with only unmodified nucleotides.

The amplification reaction may be performed in the given partition. Alternatively, the amplification reaction may be performed prior to providing the plurality of nucleic acid molecules in the plurality of partitions.

The amplification reaction may be PCR based amplification. Alternatively, the amplification may be non-PCR based amplification. For example, the amplification may be an isothermal amplification, e.g., a multiple displacement amplification (MDA) reaction. The MDA reaction may be performed by annealing random hexamer primers to the nucleic acid molecule.

The random hexamer primers may comprise a constant region. The constant region in the hexamer may be incorporated in to the nucleic acid molecule during the amplification reaction. In some cases, the constant region in the hexamer may be a recognition site for a restriction enzyme. After the attachment of the hexamer to the nucleic acid molecule, the hexamer may be used as a primer for the amplification reaction. During the amplification reaction, the hexamer may be incorporated in to the nucleic acid molecule along with the constant region. The constant region in the hexamer may then be used to fragment the nucleic acid molecule using restriction enzymes. In some cases, the hexamers may be phosphorylated at the 5′ end. The phosphorylated 5′ end of the hexamer may then be used for the ligation of the barcode or the amplified nucleic acid molecule to the hexamer.

The amplification reaction may be performed using a polymerizing enzyme. In some cases, the polymerizing enzyme may be a high fidelity polymerizing enzyme. In some examples, the polymerizing enzyme is a polymerase. Non-limiting examples of polymerases include (D29 (phi29) DNA polymerase or a functional derivative thereof, Bst DNA polymerase, and other examples presented elsewhere herein.

The amplification reaction may be performed at a temperature of about 25° C. to about 45° C., or about 25° C. to about 35° C. In some examples, the amplification reaction may be performed at a temperature of at least about 25° C., 26° C., 27° C., 28° C., 29° C., 30° C., 31° C., 32° C., 33° C. or 34° C. In some examples, the amplification reaction may be performed at a temperature of at most about 35° C., 34° C., 33° C., 32° C., 31° C., 30° C., 29° C., 28° C., 27° C. or 26° C. In some examples, the amplification reaction may be performed at a temperature of about 25° C. to about 26° C., about 25° C. to about 27° C., about 25° C. to about 28° C., about 25° C. to about 29° C., about 25° C. to about 30° C., about 25° C. to about 31° C., about 25° C. to about 32° C., about 25° C. to about 33° C., about 25° C. to about 34° C., about 25° C. to about 35° C., about 26° C. to about 27° C., about 26° C. to about 28° C., about 26° C. to about 29° C., about 26° C. to about 30° C., about 26° C. to about 31° C., about 26° C. to about 32° C., about 26° C. to about 33° C., about 26° C. to about 34° C., about 26° C. to about 35° C., about 27° C. to about 28° C., about 27° C. to about 29° C., about 27° C. to about 30° C., about 27° C. to about 31° C., about 27° C. to about 32° C., about 27° C. to about 33° C., about 27° C. to about 34° C., about 27° C. to about 35° C., about 28° C. to about 29° C., about 28° C. to about 30° C., about 28° C. to about 31° C., about 28° C. to about 32° C., about 28° C. to about 33° C., about 28° C. to about 34° C., about 28° C. to about 35° C., about 29° C. to about 30° C., about 29° C. to about 31° C., about 29° C. to about 32° C., about 29° C. to about 33° C., about 29° C. to about 34° C., about 29° C. to about 35° C., about 30° C. to about 31° C., about 30° C. to about 32° C., about 30° C. to about 33° C., about 30° C. to about 34° C., about 30° C. to about 35° C., about 31° C. to about 32° C., about 31° C. to about 33° C., about 31° C. to about 34° C., about 31° C. to about 35° C., about 32° C. to about 33° C., about 32° C. to about 34° C., about 32° C. to about 35° C., about 33° C. to about 34° C., about 33° C. to about 35° C., or about 34° C. to about 35° C. In some examples, the amplification reaction may be performed at a temperature of about 25° C., about 26° C., about 27° C., about 28° C., about 29° C., about 30° C., about 31° C., about 32° C., about 33° C., about 34° C., or about 35° C.

The amplification reaction may be performed for about 1 hour to about 3 hours. In some examples, the amplification reaction may be performed for at least about 1 hour, 2 hours, 3 hours, 4 hours, 5 hours, 6 hours, or more. In some examples, the amplification reaction may be performed for at most about 3 hours, 2 hours, 1 hours, 30 minutes, 10 minutes, or less. In some examples, the amplification reaction may be performed for about 1 hour to about 1.2 hours, about 1 hour to about 1.5 hours, about 1 hour to about 1.7 hours, about 1 hour to about 2 hours, about 1 hour to about 2.2 hours, about 1 hour to about 2.5 hours, about 1 hour to about 2.7 hours, about 1 hour to about 3 hours, about 1.2 hours to about 1.5 hours, about 1.2 hours to about 1.7 hours, about 1.2 hours to about 2 hours, about 1.2 hours to about 2.2 hours, about 1.2 hours to about 2.5 hours, about 1.2 hours to about 2.7 hours, about 1.2 hours to about 3 hours, about 1.5 hours to about 1.7 hours, about 1.5 hours to about 2 hours, about 1.5 hours to about 2.2 hours, about 1.5 hours to about 2.5 hours, about 1.5 hours to about 2.7 hours, about 1.5 hours to about 3 hours, about 1.7 hours to about 2 hours, about 1.7 hours to about 2.2 hours, about 1.7 hours to about 2.5 hours, about 1.7 hours to about 2.7 hours, about 1.7 hours to about 3 hours, about 2 hours to about 2.2 hours, about 2 hours to about 2.5 hours, about 2 hours to about 2.7 hours, about 2 hours to about 3 hours, about 2.2 hours to about 2.5 hours, about 2.2 hours to about 2.7 hours, about 2.2 hours to about 3 hours, about 2.5 hours to about 2.7 hours, about 2.5 hours to about 3 hours, or about 2.7 hours to about 3 hours. In some examples, the amplification reaction may be performed for about 1 hour, about 1.2 hours, about 1.5 hours, about 1.7 hours, about 2 hours, about 2.2 hours, about 2.5 hours, about 2.7 hours, or about 3 hours.

The amplification reaction may be performed in the presence of buffer solutions. Any suitable buffer may be used for the amplification reaction. The buffer solutions may comprise salts. In some examples, the buffer solutions comprise potassium salts, e.g., potassium glutamate.

The nucleotide analogs used in the amplification reactions may be methylated nucleotide analogs. In some examples, the mixture of nucleotide bases comprises a mix of unmodified nucleotide bases and methylated nucleotide bases. In some examples, the nucleotide mixture used in the amplification reaction comprises one type of nucleotide analog and other unmodified nucleotide analogs. For example, the reaction mixture may comprise methylated cytosine nucleotide analogs in combination with a mix of naturally occurring dNTPs. In this example, the amplification reaction may comprise naturally occurring cytosine nucleotide base in addition to methylated cytosine nucleotide bases. Alternatively, it may only comprise methylated cytosine in a mixture with naturally occurring thymine, adenine and guanine nucleotide bases.

A given partition may comprise modification dependent restriction enzymes. The modification dependent restriction enzyme may be a modification dependent restriction endonuclease. In some cases, the modification dependent restriction endonuclease is a methylation dependent restriction endonuclease. Any suitable modification dependent restriction enzyme may be used, examples of which are presented elsewhere herein. For example, a methylation dependent restriction enzyme may be MspJI. Alternatively, a partition comprising the given nucleic acid molecule may be merged with a partition comprising reagents, such as the modification dependent restriction enzyme.

The modification dependent restriction enzyme may have a specific recognition site. The recognition site may include the nucleotide analog. In some cases, the restriction enzyme may only fragment the nucleic acid molecule in the presence of a nucleotide analog. The modification dependent restriction enzyme may fragment the nucleic acid molecule at the location of the nucleotide analog. Alternatively, the modification dependent restriction enzyme may fragment the nucleic acid molecule at a position proximal to the nucleotide analog.

In some examples, the modification dependent restriction enzyme fragments the nucleic acid molecule at a position up to 1 nucleotide base from the nucleotide analog. In other examples, the modification dependent restriction enzyme may fragment the nucleic acid molecule at a position up to 2, 3, 4, 5, 6, 7, 8, 9 or 10 bases from the nucleotide analog.

The restriction enzyme may be a blunt cutter that yields a blunt ended fragmented nucleic acid molecule with no overhangs. Alternatively, the restriction enzyme may be a cutter that yields a single stranded overhang. A single stranded overhang may act as a cohesive end and be used to barcode a nucleic acid molecules. A single stranded overhang may be modified to result in a blunt end.

A given partition may comprise restriction enzymes that are not modification dependent. These restriction enzymes may be used to fragment the nucleic acid molecule without the presence of a nucleotide analog. A partition comprising the given nucleic acid molecule may be merged with a partition comprising the restriction enzyme. Any suitable restriction enzyme may be used, examples of which are presented elsewhere herein.

The fragmentation of the nucleic acid molecule may be performed by a plurality of restriction enzymes. For instance, the nucleic acid molecule may be fragmented by two or more different restriction enzymes. The two or more restriction enzymes may be used in the same reaction. In some examples, one of the restriction enzymes may be a modification dependent restriction endonuclease and one of the restriction enzymes may be a restriction endonuclease that is not modification dependent. For instance, MspJI, a methylation dependent restriction endonuclease may be used in combination with other restriction endonucleases, e.g., AluI or RsaI or any other restriction enzymes presented elsewhere herein.

The fragmentation of the nucleic acid molecules may be performed by restriction enzymes that are not modification dependent. For example, if the amplification reaction was performed with only unmodified nucleotides, restriction endonucleases that fragment nucleic acids with only naturally occurring or unmodified nucleotides may be used for fragmentation. In some cases, a plurality of restriction enzymes that are not modification dependent may be used to fragment the nucleic acid molecules. For example, restriction endonucleases such as AluI, RsaI may be used in combination to fragment the nucleic acid molecules. Other suitable restriction enzymes, listed elsewhere herein may also be used in fragmentation reactions.

The fragmentation of the nucleic acid molecule may be performed in a given partition. The fragmentation of the nucleic acid molecule by the restriction enzyme may be performed at a temperature of about 15° C. to about 75° C. In some examples, the fragmentation of the nucleic acid molecule by the restriction enzyme may be performed at a temperature of about 30° C. to about 40° C. In some examples, the fragmentation of the nucleic acid molecule by the restriction enzyme may be performed at a temperature of at least about 30° C., 31° C., 32° C., 33° C., 34° C., 35° C., 36° C., 37° C., 38° C. or 39° C. In some examples, the restriction enzyme may be active at a temperature of at most about 40° C., 39° C., 38° C., 37° C., 36° C., 35° C., 34° C., 33° C., 32° C. or 31° C. In some examples, fragmentation of the nucleic acid molecule by the restriction enzyme may be performed at a temperature of about 30° C. to about 31° C., about 30° C. to about 32° C., about 30° C. to about 33° C., about 30° C. to about 34° C., about 30° C. to about 35° C., about 30° C. to about 36° C., about 30° C. to about 37° C., about 30° C. to about 38° C., about 30° C. to about 39° C., about 30° C. to about 40° C., about 31° C. to about 32° C., about 31° C. to about 33° C., about 31° C. to about 34° C., about 31° C. to about 35° C., about 31° C. to about 36° C., about 31° C. to about 37° C., about 31° C. to about 38° C., about 31° C. to about 39° C., about 31° C. to about 40° C., about 32° C. to about 33° C., about 32° C. to about 34° C., about 32° C. to about 35° C., about 32° C. to about 36° C., about 32° C. to about 37° C., about 32° C. to about 38° C., about 32° C. to about 39° C., about 32° C. to about 40° C., about 33° C. to about 34° C., about 33° C. to about 35° C., about 33° C. to about 36° C., about 33° C. to about 37° C., about 33° C. to about 38° C., about 33° C. to about 39° C., about 33° C. to about 40° C., about 34° C. to about 35° C., about 34° C. to about 36° C., about 34° C. to about 37° C., about 34° C. to about 38° C., about 34° C. to about 39° C., about 34° C. to about 40° C., about 35° C. to about 36° C., about 35° C. to about 37° C., about 35° C. to about 38° C., about 35° C. to about 39° C., about 35° C. to about 40° C., about 36° C. to about 37° C., about 36° C. to about 38° C., about 36° C. to about 39° C., about 36° C. to about 40° C., about 37° C. to about 38° C., about 37° C. to about 39° C., about 37° C. to about 40° C., about 38° C. to about 39° C., about 38° C. to about 40° C., or about 39° C. to about 40° C. In some examples, the fragmentation of the nucleic acid molecule by the restriction enzyme may be performed at a temperature of about 30° C., about 31° C., about 32° C., about 33° C., about 34° C., about 35° C., about 36° C., about 37° C., about 38° C., about 39° C., or about 40° C.

In some examples, the fragmentation reaction may be performed for about 10 minutes to about 30 minutes. In some examples, the fragmentation reaction may be performed for at least about 10 minutes. In some examples, the fragmentation reaction may be performed for at most about 30 minutes. In some examples, the fragmentation reaction may be performed for about 10 minutes to about 12 minutes, about 10 minutes to about 15 minutes, about 10 minutes to about 17 minutes, about 10 minutes to about 20 minutes, about 10 minutes to about 22 minutes, about 10 minutes to about 25 minutes, about 10 minutes to about 27 minutes, about 10 minutes to about 30 minutes, about 12 minutes to about 15 minutes, about 12 minutes to about 17 minutes, about 12 minutes to about 20 minutes, about 12 minutes to about 22 minutes, about 12 minutes to about 25 minutes, about 12 minutes to about 27 minutes, about 12 minutes to about 30 minutes, about 15 minutes to about 17 minutes, about 15 minutes to about 20 minutes, about 15 minutes to about 22 minutes, about 15 minutes to about 25 minutes, about 15 minutes to about 27 minutes, about 15 minutes to about 30 minutes, about 17 minutes to about 20 minutes, about 17 minutes to about 22 minutes, about 17 minutes to about 25 minutes, about 17 minutes to about 27 minutes, about 17 minutes to about 30 minutes, about 20 minutes to about 22 minutes, about 20 minutes to about 25 minutes, about 20 minutes to about 27 minutes, about 20 minutes to about 30 minutes, about 22 minutes to about 25 minutes, about 22 minutes to about 27 minutes, about 22 minutes to about 30 minutes, about 25 minutes to about 27 minutes, about 25 minutes to about 30 minutes, or about 27 minutes to about 30 minutes. In some examples, the fragmentation reaction may be performed for about 10 minutes, about 12 minutes, about 15 minutes, about 17 minutes, about 20 minutes, about 22 minutes, about 25 minutes, about 27 minutes, or about 30 minutes.

The fragmented nucleic acid may comprise fragments in a size range from about 10 to 1000 nucleotide bases. In some examples, the fragmented nucleic acid may comprise fragments in a size range of at least about 10, 20, 30, 40, 50, 100, 200, 300, 400, 500, 600, 700, 800, 900, 950 or 990 nucleotide bases. In some examples, the fragmented nucleic acid may comprise fragments in a size range of at most about 1000, 990, 950, 900, 800, 700, 600, 500, 400, 300, 200, 100, 50, 40, 30 or 20 nucleotide bases. In some examples, the fragmented nucleic acid may comprise fragments in a size range of about 10 nucleotide bases to about 20 nucleotide bases, about 10 nucleotide bases to about 30 nucleotide bases, about 10 nucleotide bases to about 40 nucleotide bases, about 10 nucleotide bases to about 50 nucleotide bases, about 10 nucleotide bases to about 100 nucleotide bases, about 10 nucleotide bases to about 200 nucleotide bases, about 10 nucleotide bases to about 300 nucleotide bases, about 10 nucleotide bases to about 500 nucleotide bases, about 10 nucleotide bases to about 700 nucleotide bases, about 10 nucleotide bases to about 800 nucleotide bases, about 10 nucleotide bases to about 1000 nucleotide bases, about 20 nucleotide bases to about 30 nucleotide bases, about 20 nucleotide bases to about 40 nucleotide bases, about 20 nucleotide bases to about 50 nucleotide bases, about 20 nucleotide bases to about 100 nucleotide bases, about 20 nucleotide bases to about 200 nucleotide bases, about 20 nucleotide bases to about 300 nucleotide bases, about 20 nucleotide bases to about 500 nucleotide bases, about 20 nucleotide bases to about 700 nucleotide bases, about 20 nucleotide bases to about 800 nucleotide bases, about 20 nucleotide bases to about 1000 nucleotide bases, about 30 nucleotide bases to about 40 nucleotide bases, about 30 nucleotide bases to about 50 nucleotide bases, about 30 nucleotide bases to about 100 nucleotide bases, about 30 nucleotide bases to about 200 nucleotide bases, about 30 nucleotide bases to about 300 nucleotide bases, about 30 nucleotide bases to about 500 nucleotide bases, about 30 nucleotide bases to about 700 nucleotide bases, about 30 nucleotide bases to about 800 nucleotide bases, about 30 nucleotide bases to about 1000 nucleotide bases, about 40 nucleotide bases to about 50 nucleotide bases, about 40 nucleotide bases to about 100 nucleotide bases, about 40 nucleotide bases to about 200 nucleotide bases, about 40 nucleotide bases to about 300 nucleotide bases, about 40 nucleotide bases to about 500 nucleotide bases, about 40 nucleotide bases to about 700 nucleotide bases, about 40 nucleotide bases to about 800 nucleotide bases, about 40 nucleotide bases to about 1000 nucleotide bases, about 50 nucleotide bases to about 100 nucleotide bases, about 50 nucleotide bases to about 200 nucleotide bases, about 50 nucleotide bases to about 300 nucleotide bases, about 50 nucleotide bases to about 500 nucleotide bases, about 50 nucleotide bases to about 700 nucleotide bases, about 50 nucleotide bases to about 800 nucleotide bases, about 50 nucleotide bases to about 1000 nucleotide bases, about 100 nucleotide bases to about 200 nucleotide bases, about 100 nucleotide bases to about 300 nucleotide bases, about 100 nucleotide bases to about 500 nucleotide bases, about 100 nucleotide bases to about 700 nucleotide bases, about 100 nucleotide bases to about 800 nucleotide bases, about 100 nucleotide bases to about 1000 nucleotide bases, about 200 nucleotide bases to about 300 nucleotide bases, about 200 nucleotide bases to about 500 nucleotide bases, about 200 nucleotide bases to about 700 nucleotide bases, about 200 nucleotide bases to about 800 nucleotide bases, about 200 nucleotide bases to about 1000 nucleotide bases, about 300 nucleotide bases to about 500 nucleotide bases, about 300 nucleotide bases to about 700 nucleotide bases, about 300 nucleotide bases to about 800 nucleotide bases, about 300 nucleotide bases to about 1000 nucleotide bases, about 500 nucleotide bases to about 700 nucleotide bases, about 500 nucleotide bases to about 800 nucleotide bases, about 500 nucleotide bases to about 1000 nucleotide bases, about 700 nucleotide bases to about 800 nucleotide bases, about 700 nucleotide bases to about 1000 nucleotide bases, or about 800 nucleotide bases to about 1000 nucleotide bases. In some examples, the fragmented nucleic acid may comprise fragments in a size range of about 10 nucleotide bases, about 20 nucleotide bases, about 30 nucleotide bases, about 40 nucleotide bases, about 50 nucleotide bases, about 100 nucleotide bases, about 200 nucleotide bases, about 300 nucleotide bases, about 500 nucleotide bases, about 700 nucleotide bases, about 800 nucleotide bases, about 900 nucleotide bases or about 1000 nucleotide bases.

The fragmentation reaction may be followed by an end-repair reaction. The end-repair reaction may be performed for creating a blunt ended fragment or to phosphorylate the nucleotide base ends for subsequent ligation. The end repair reaction may be performed in the presence of T4 DNA polymerase or Klenow or a functional fragment thereof.

The fragmented nucleic acid molecule may be subjected to adaptor ligation reactions. The adaptors ligated to the fragmented nucleic acid molecule may be used for sequencing the fragments. Adapters used may be commercially available adapters for example, Illumina TruSeq®, Roche® NimbleGen™ SegCap™ EZ, Agilent® SureSelect, etc.

The oligonucleotide barcode molecule may comprise a barcode sequence and may be used to barcode a given nucleic acid fragment from the plurality of nucleic acid fragments. This may yield a barcoded fragment comprising the barcode sequence. The barcoding of the nucleic acid fragments may be performed in the given partition comprising the fragmented nucleic acid molecules.

Alternatively, the fragmentation may be performed in a separate partition than the barcoding. For example, nucleic acid molecules from a cell or cell bead may be fragmented in a first partition. A bead with oligonucleotide barcode molecules may then be introduced in this partition—e.g., by merging a second partition having the bead with the first partition. The barcoding of the fragmented nucleic acid molecules may then be performed. The partitions may be droplets or wells.

The nucleic acid barcode sequences may be attached to the nucleic acid molecules or the fragments of the nucleic acid molecules by ligation. The ligation may be performed with a ligase enzyme, for example a T4 DNA ligase or a functional derivative of the T4 DNA ligase. Other suitable ligase enzymes such as thermostable ligases may be used.

The barcode molecules may be attached to the hexamer incorporated into the nucleic acid molecule. In some cases, the sequence of the barcode molecules may be complementary to the hexamer sequence.

The ligation of the barcodes to the fragmented nucleic acid molecules may be performed at a temperature of about 16° C. to about 30° C. In some examples, the ligation may be performed at a temperature of at least about 16° C., 17° C., 18° C., 19° C., 20° C., 21° C., 22° C., 23° C., 24° C., 25° C., 26° C., 27° C., 28° C. or 29° C. In some examples, the ligation may be performed at a temperature of at most about 30° C., 29° C., 27° C., 25° C., 24° C., 23° C., 22° C., 21° C., 20° C., 19° C., 18° C. or 17° C. In some examples, the ligation may be performed at a temperature of about 16° C. to about 17° C., about 16° C. to about 18° C., about 16° C. to about 19° C., about 16° C. to about 20° C., about 16° C. to about 21° C., about 16° C. to about 22° C., about 16° C. to about 23° C., about 16° C. to about 24° C., about 16° C. to about 25° C., about 16° C. to about 27° C., about 16° C. to about 30° C., about 17° C. to about 18° C., about 17° C. to about 19° C., about 17° C. to about 20° C., about 17° C. to about 21° C., about 17° C. to about 22° C., about 17° C. to about 23° C., about 17° C. to about 24° C., about 17° C. to about 25° C., about 17° C. to about 27° C., about 17° C. to about 30° C., about 18° C. to about 19° C., about 18° C. to about 20° C., about 18° C. to about 21° C., about 18° C. to about 22° C., about 18° C. to about 23° C., about 18° C. to about 24° C., about 18° C. to about 25° C., about 18° C. to about 27° C., about 18° C. to about 30° C., about 19° C. to about 20° C., about 19° C. to about 21° C., about 19° C. to about 22° C., about 19° C. to about 23° C., about 19° C. to about 24° C., about 19° C. to about 25° C., about 19° C. to about 27° C., about 19° C. to about 30° C., about 20° C. to about 21° C., about 20° C. to about 22° C., about 20° C. to about 23° C., about 20° C. to about 24° C., about 20° C. to about 25° C., about 20° C. to about 27° C., about 20° C. to about 30° C., about 21° C. to about 22° C., about 21° C. to about 23° C., about 21° C. to about 24° C., about 21° C. to about 25° C., about 21° C. to about 27° C., about 21° C. to about 30° C., about 22° C. to about 23° C., about 22° C. to about 24° C., about 22° C. to about 25° C., about 22° C. to about 27° C., about 22° C. to about 30° C., about 23° C. to about 24° C., about 23° C. to about 25° C., about 23° C. to about 27° C., about 23° C. to about 30° C., about 24° C. to about 25° C., about 24° C. to about 27° C., about 24° C. to about 30° C., about 25° C. to about 27° C., about 25° C. to about 30° C., or about 27° C. to about 30° C. In some examples, the ligation may be performed at a temperature of about 16° C., about 17° C., about 18° C., about 19° C., about 20° C., about 21° C., about 22° C., about 23° C., about 24° C., about 25° C., about 27° C., or about 30° C.

The ligation reaction may be performed for about 0.5 hours to about 2 hours. In some examples, the ligation reaction may be performed for at least about 0.5 hours. In some examples, the ligation reaction may be performed for at most about 2 hours. In some examples, the ligation reaction may be performed for about 0.5 hours to about 0.6 hours, about 0.5 hours to about 0.7 hours, about 0.5 hours to about 0.8 hours, about 0.5 hours to about 1 hours, about 0.5 hours to about 1.2 hours, about 0.5 hours to about 1.5 hours, about 0.5 hours to about 1.7 hours, about 0.5 hours to about 2 hours, about 0.6 hours to about 0.7 hours, about 0.6 hours to about 0.8 hours, about 0.6 hours to about 1 hours, about 0.6 hours to about 1.2 hours, about 0.6 hours to about 1.5 hours, about 0.6 hours to about 1.7 hours, about 0.6 hours to about 2 hours, about 0.7 hours to about 0.8 hours, about 0.7 hours to about 1 hours, about 0.7 hours to about 1.2 hours, about 0.7 hours to about 1.5 hours, about 0.7 hours to about 1.7 hours, about 0.7 hours to about 2 hours, about 0.8 hours to about 1 hours, about 0.8 hours to about 1.2 hours, about 0.8 hours to about 1.5 hours, about 0.8 hours to about 1.7 hours, about 0.8 hours to about 2 hours, about 1 hours to about 1.2 hours, about 1 hours to about 1.5 hours, about 1 hours to about 1.7 hours, about 1 hours to about 2 hours, about 1.2 hours to about 1.5 hours, about 1.2 hours to about 1.7 hours, about 1.2 hours to about 2 hours, about 1.5 hours to about 1.7 hours, about 1.5 hours to about 2 hours, or about 1.7 hours to about 2 hours. In some examples, the ligation reaction may be performed for about 0.5 hours, about 0.6 hours, about 0.7 hours, about 0.8 hours, about 1 hour, about 1.2 hours, about 1.5 hours, about 1.7 hours, or about 2 hours.

The nucleic acid barcode sequences may be attached to the nucleic acid molecules or the fragments of the nucleic acid molecules by transposons. The primers or random hexamers used to incorporate nucleotides or nucleotide analogs in to the nucleic acid molecule may comprise a sequence complementary to transposon end sequences. The partition (e.g., droplet) comprising the fragmented nucleic acid molecule may include nucleic acid barcode molecules comprising barcode sequences and transposase molecule(s). The barcode sequences may comprise the transposon end sequences. The transposase molecule(s) may then be used to attach the barcode sequence to the fragmented nucleic acid molecule.

Nucleic acid molecules may be provided by way of cells or cell beads, for example. The cells or cell beads may be included in partitions and nucleic acid molecules may be released from the cells or cell beads in the partitions. Next, the nucleic acid molecules may be fragmented. The fragmented nucleic acid molecules may be barcoded in the partitions using, for example, nucleic acid barcode molecules coupled to beads. A partition can include a single cell or cell bead and a single bead.

The oligonucleotide barcode molecule may be from a plurality of oligonucleotide barcode molecules. In some cases, the plurality of oligonucleotide barcode molecules may comprise first barcode sequences and second barcode sequences. The first barcode sequences used may be the same across the plurality of oligonucleotide barcode molecules. The second barcode sequences used may be different across the plurality of oligonucleotide barcode molecules.

The barcoded nucleic acid fragments may be subjected to conditions to remove the barcoded fragment from the partition. In some cases, the partition may be a droplet. The partition may be a droplet as part of an emulsion that may comprise a plurality of droplets. The removal of the barcoded fragment may comprise disrupting the emulsion to release the barcoded fragment or a derivative thereof from the given partition. The removal of the barcoded nucleic acid fragment may be performed by any suitable approach, including examples provided elsewhere herein.

The partitioned barcoded fragmented nucleic acid molecule may be subjected to one or more reactions after the barcoding. In some cases, the partitioned barcoded fragment or derivative thereof may be subjected to nucleic acid amplification. The amplification may be performed upon removing the barcoded fragment or derivative thereof from the given partition. In some cases, the nucleic acid amplification is polymerase chain reaction (PCR). In some cases, the nucleic acid amplification is isothermal amplification (e.g., MDA). In some cases, the amplification comprises thermal cycling.

In some cases, these reactions may be performed in a given partition. For example, the nucleic acid amplification may be performed in the same partition where the barcoding reaction was performed. Alternatively, these reactions may be performed external to the given partition. For example, the nucleic acid amplification may be performed in bulk with the barcoded nucleic acid fragments from other partitions.

In some cases, the one or more reactions may comprise coupling a flow cell sequence to the barcoded fragment or derivative thereof. The flow cell sequence may permit the attachment of the barcoded fragment or derivative thereof to a flow cell of a sequencer.

The barcoded fragment or derivative thereof may be subjected to nucleic acid sequencing. The sequencing may be performed to identify a sequence of at least a portion of a given nucleic acid fragment and at least a portion of the barcode sequence.

The plurality of partitions may be a plurality of wells. Alternatively, the plurality of partitions may be a plurality of droplets.

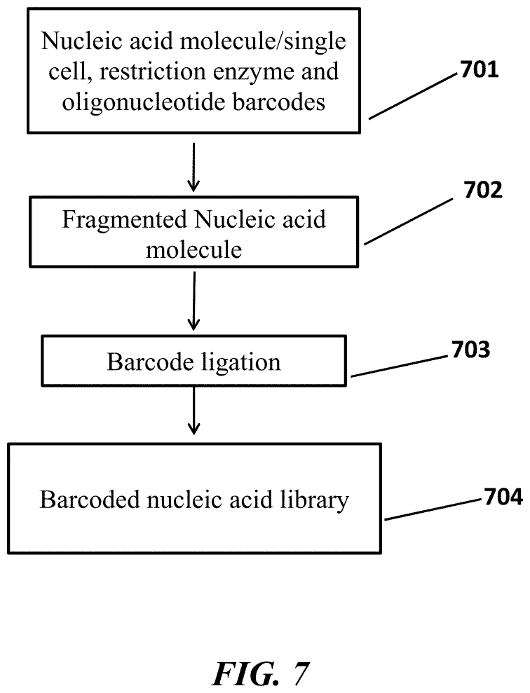

Various example schemes are described below. In an example, a nucleic acid molecule may be processed in discrete partitions (e.g., droplets). FIG. 7 shows a method for processing a nucleic acid molecule. In a first operation 701, a partition comprising the nucleic acid molecule is provided. The partition may include a bead comprising nucleic acid barcode molecules coupled thereto. Barcodes may be in the form of unique oligonucleotides. The partition may also comprise a restriction enzyme. In a second operation 702 the restriction enzyme may be used to fragment the nucleic acid molecule. If the nucleic acid molecule is in a cell or cell bead in the partition, for example, the nucleic acid molecule may be released from the cell or cell bead. Alternatively, the nucleic acid molecule is fragmented in the cell or cell bead and the fragments are subsequently released.

Fragmentation may be performed by subjecting the partition comprising a given nucleic acid molecule and a given restriction enzyme to temperatures that activate the restriction enzyme. In a third operation 703, upon or during fragmentation, a nucleic acid barcode molecule from the bead may be used to barcode a fragment of the nucleic acid molecule to yield a barcoded fragment. The nucleic acid barcode molecule may be ligated to the fragment of the nucleic acid molecule, for example. Other nucleic acid fragments in the partition may be barcoded, thereby generating a barcoded nucleic acid library, as shown in operation 704. The resulting barcoded fragments or the barcoded nucleic acid library in the partition may be subjected to further processing, e.g., sequencing.

In one example, the nucleic acid molecule may comprise a methylated nucleotide analog. For instance, this nucleotide analog may be a methylated cytosine. Methylated cytosine may be randomly incorporated in to the nucleic acid molecule during an amplification reaction. In this case, the restriction enzyme may be a methylation dependent restriction enzyme, e.g., MspJI. The partition comprising the nucleic acid molecules (in this case with a methylated cytosine incorporated randomly) and the restriction enzyme MspJI may be subjected to temperatures that activate MspJI. Upon activation, MspJI recognizes the methylated cytosine and may fragment the nucleotide molecule into at least two fragments in the partition. Upon fragmentation, these fragmented molecules may be barcoded using nucleic acid barcode molecules. The nucleic acid barcode molecules may have sample indexes and/or unique molecule identifiers.

Barcoding may be performed via ligation. In such a case, a partition may include a ligation enzyme (e.g., ligase) and the ligation enzyme and the nucleic acid barcode molecules may be subjected to temperatures sufficient to facilitate a ligation reaction in which a nucleic acid barcode molecule is ligated to a nucleic acid fragment. This may yield a library of barcoded fragmented nucleic acid molecules. This library of barcoded fragmented nucleic acid molecules may then be processed in bulk with other barcoded fragments and subjected to sequencing.

### Computer Systems

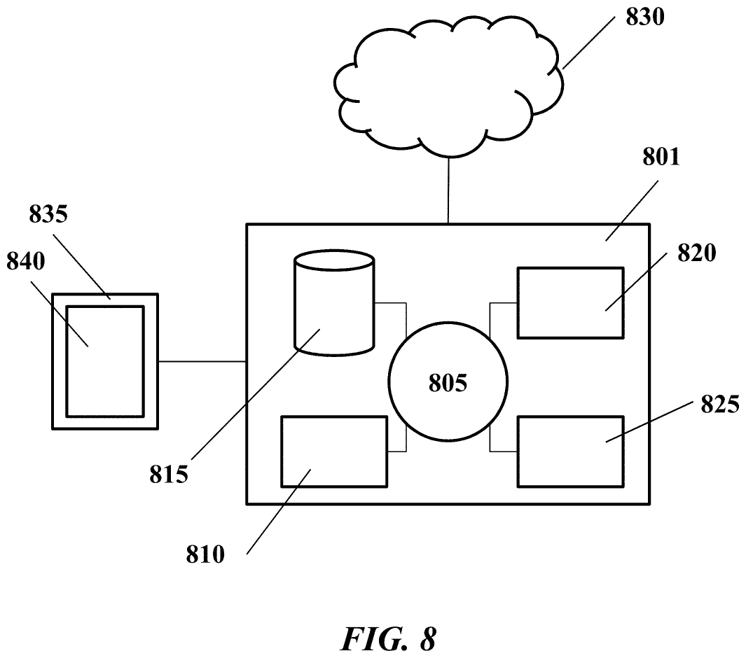

The present disclosure provides computer systems that are programmed to implement methods of the disclosure. FIG. 8 shows a computer system 801 that is programmed or otherwise configured to (i) control a microfluidics system (e.g., fluid flow), (ii) control the reaction temperatures during different cycles, (iii) polymerize droplets, (iv) perform sequencing applications, (v) generate and maintain a library of nucleic acid molecules and (vi) analyze sequences. The computer system 801 can regulate various aspects of the present disclosure, such as, for example, regulating fluid flow rate in one or more channels in a microfluidic structure, regulating the temperature changes during different reaction cycles, regulating polymerization application units, etc. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, results of sequencing reactions. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, e.g., control the reaction temperatures and times in different cycles, perform sequencing, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) form a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

### EXAMPLES

### Example 1

Genomic DNA from Jurkat cells and mouse EL4 cells was subjected to an amplification reaction in the presence of methylated dCTPs for random incorporation of the methylated dCTPs in to the amplified DNA. The amplification reaction was followed by a restriction endonuclease reaction. Reaction mixtures comprising DNA polymerases, methylation dependent restriction enzymes and genomic DNA were incubated for 3 hours at 30° C. and then 60 minutes at 37° C. Then the reaction was incubated at 16° C. for 3 hours then at 65° C. for 10 minutes and stored at 4° C.

TABLE 1
Reaction mixture for amplification reaction.

| | | | | | Z2, 1 | Z2 |
|---|---|---|---|---|---|---|
| | | | Conc | | Rxn | MasterMix |
| Z2 Components | Stock Conc. | | [in GEM] | | (μl) | (μl) |
| Ambion | — | | — | | 30.1 | 265.0 |
| Nuclease- | | | | | | |
| Free Water | | | | | | |
| Phi29 buffer | 10X | | 1X | | 8.5 | 74.8 |
| MgC12 | 1000 | mM | 6 | mM | 0.8 | 6.9 |
| Surfactant | 10% | | 0.5% | | 4.3 | 37.4 |
| dNTP | 10 | mM | 1.000 | mM | 13.1 | 115.2 |
| mdCTP | 1 | mM | 0.050 | mM | 6.5 | |
| Hexamer PP622 | 1000 | uM | 50 | uM | 6.5 | 57.6 |
| (40% GC) | | | | | | |
| ATP | 100 | mM | 1 | mM | 1.3 | 11.5 |
| Glycerol | 50% | | 7% | | 0.8 | 6.8 |
| DTT | 1000 | mM | 15 | mM | 2.0 | 17.3 |
| Phi29 | 70.57 | uM | 2 | uM | 3.7 | 32.6 |
| T4 DNA Ligase | 300 | U/μl | 2 | U/μl | 0.9 | 7.7 |
| MspJI | 10 | U/μl | 0.5 | U/μl | 6.5 | |
| | Stock 5 | U/μl | 0.25 | U/μl | 85.0 | 71.9 |

The digested fragments were then end repaired. The sample reactions were brought up to 50 μl. The digested fragments were end repaired according to the reaction mixtures in Tables 2A-2B.

TABLE 2A
End repair reaction mixture

| | | | 8 Libraries | 7 Libraries |
|---|---|---|---|---|
| End Repair Mastermix | 1 Library | | (5% overage) | (5% overage) |
| 8.5X KAPA End Repair | 7 | μl | 59 μl | 51 μl |
| & A-tailing Buffer | | | | |
| KAPA End Repair & | 3 | μl | 25 μl | 22 μl |
| A-tailing Enzyme Mix | | | | |
| Totla mastermix volume | 10 | μl | 84 μl | 74 μl |

TABLE 2B
End repair reaction conditions

| End Repair reaction | | |
|---|---|---|
| Fragmented, dsDNA | 50 | μl |
| End Repair Mastermix | 10 | μl |
| Total reaction volume | 60 | μl |
| Incubate with the following protocol: | | |
| Temperature | Time | |
| 20° C. | 30 | min |
| 65° C. | 30 | min |

| | |
|---|---|
| 4° C. | Hold |

The digested fragments were then subjected to adaptor ligation reaction using a mixture as presented in Table 3.

TABLE 3
Adaptor ligation reaction mixture

| A-Tailing | | 8 Libraries | 7 Libraries |
|---|---|---|---|
| Mastermix | 1 Library | (5% overage) | (5% overage) |
| Water | 7.5 μl | 63 μl | 55 μl |
| 3.7X KAPA | 30 μl | 252 μl | 221 μl |
| Ligation Buffer | | | |
| KAPA DNA Ligase | 10 μl | 84 μl | 74 μl |
| 30 uM Universal | 2.5 μl | 21.0 μl | 18.4 μl |
| Adaptor | | | |
| Total mastermix | 50 μl | 420 μl | 368 μl |
| volume | | | |

50 μl ligation mastermix was added to 60 μl of End repair& A-Tailing reaction product and incubated at room temperature for 15 minutes. The reaction was then subjected to an adaptor ligation cleanup reaction using Solid Phase Reversible Immobilisation (SPRI) beads to reversibly bind DNA fragments to remove any remaining unbound adaptors and to concentrate DNA fragments in the library as in Table 4. SPRI beads were added to the ligation reaction product. The reaction mixture was incubated for 5 minutes at room temperature. Post incubation, the supernatant was removed and the reaction mixture was washed twice with 200 μl 80% EtOH. Beads were allowed to dry for 2-3 minutes. Beads were eluted in 50 μl elution buffer.

TABLE 4
Adaptor ligation cleanup reaction

| | |
|---|---|
| Adaptor ligation reaction product | 110 μl |
| SPRI beads | 88 μl |
| Total Volume per well/tube | 198 μl |

The barcoded library was then subjected to another amplification reaction using a mastermix and conditions as shown in Tables 5A-C.

TABLE 5A
Library amplification reaction mastermix

| | | 7 Libraries |
|---|---|---|
| Library Amplification Mastermix | 1 Library | (5% overage) |
| 2X KAPA HiFi U + Master Mix | 50 μl | 368 μl |
| 10 uM PP695 Primer | 5.0 μl | 37 μl |

TABLE 5B
Library amplification reaction

| Library Amplification Reaction | |
|---|---|
| Library DNA | 40 μl |
| Library Amplification Mastermix | 55 μl |
| SI primer | 5 μl |
| Total Volume per well/tube | 100 μl |

TABLE 5C
Library amplification reaction conditions
Thermocycling

| Step | Temp | Cycles |
|---|---|---|
| Initial Denaturation | 98° C. | 1 |
| Denaturation | 98° C. | repeat 11X |
| Annealing | 54° C. | |
| Extension | 72° C. | |
| Final Extension | 72° C. | 1 |
| Stop Reaction | 4° C. | Hold |

After the amplification reaction, the library was subjected to another clean up reaction using a PCR reaction clean up kit. The samples were then subjected to sequencing using an Illumina sequencer. In the test samples the mean duplication rate was found to be low (5.5%) which indicated a high library complexity. The unmapped fraction was found to be low (<15%) and >90% reads were found in single cells. The library complexity was around 5 million unique molecules.

### Example 2

In this example, genomic DNA from Jurkat cells and mouse EL4 cells was subjected to an amplification reaction followed by a restriction endonuclease digestion reaction. The restriction enzymes used in this example are AluI and RsaI used in combination. These enzymes are not methylation dependent and thus do not require methylated dCTPs to be integrated in to the nucleic acid molecule. Reaction mixtures were made as presented in Table 6. The mastermix was divided into 4 reactions and incubated for 3 hours at 30° C. and then 60 minutes at 37° C. Then the reaction was incubated at 16° C. for 3 hours then at 65° C. for 10 minutes and stored at 4° C.

TABLE 6
Reaction mixture for amplification reaction.

| | | | | | Z2, 1 | Z2 |
|---|---|---|---|---|---|---|
| | Stock | | Conc | | Rxn | MasterMix |
| Z2 Components | Conc. | | [in GEM] | | (μl) | (μl) |
| Ambion Nuclease- | — | | — | | 36.7 | 362.9 |
| Free Water | | | | | | |
| Phi29 buffer | 10X | | 1X | | 8.5 | 84.2 |
| MgCl2 | 1000 | mM | 6 | mM | 0.8 | 7.8 |
| Surfactant | 10% | | 0.5% | | 4.3 | 42.1 |
| dNTP | 10 | mM | 1.000 | mM | 13.1 | 129.6 |
| Hexamer PP622 | 1000 | uM | 50 | uM | 6.5 | 64.8 |
| (40% GC) | | | | | | |
| ATP | 100 | mM | 1 | mM | 1.3 | 13.0 |
| Glycerol | 50% | | 7% | | 6.3 | 62.1 |
| DTT | 1000 | mM | 15 | mM | 2.0 | 19.4 |
| Phi29 | 70.57 | uM | 2 | uM | 3.7 | 36.7 |
| T4 DNA Ligase | 300 | U/μl | 2 | U/μl | 0.9 | 8.6 |
| AluI and RsaI | 5 | U/μl | 0.04 | U/μl | 1.0 | |

| | | |
|---|---|---|
| Stock 5 U/μl | 85.0 | 84.0 |

The digested fragments were then end repaired. The sample reactions were brought up to 50 μl. The digested fragments were end repaired according to the reaction mixtures in Tables 7A-7C.

TABLE 7A
End repair reaction mastermix

| | | 8 Libraries | 7 Libraries |
|---|---|---|---|
| End Repair Mastermix | 1 Library | (5% overage) | (5% overage) |
| 8.5X KAPA End Repair & | 7 μl | 59 μl | 125 μl |
| A-tailing Buffer | | | |
| KAPA End Repair & | 3 μl | 25 μl | 54 μl |
| A-tailing Enzyme Mix | | | |
| Total mastermix volume | 10 μl | 84 μl | 179 μl |

TABLE 7B
End repair reaction mixture

| End Repair reaction | |
|---|---|
| Fragmented, dsDNA | 50 μl |
| End Repair Mastermix | 10 μl |
| Total reaction volume | 60 μl |

TABLE 7 C
End repair reaction conditions

| Incubate with the following protocol: | |
|---|---|
| Temperature | Time |
| 20° C. | 30 min |
| 65° C. | 30 min |
| 4° C. | Hold |

The digested fragments were then subjected to adaptor ligation reaction as presented in Table 8.

TABLE 8
Adaptor ligation reaction mixture.

| | | 8 Libraries | 7 Libraries |
|---|---|---|---|
| A-Tailing Mastermix | 1 Library | (5% overage) | (5% overage) |
| Water | 7.5 μl | 63 μl | 134 μl |
| 3.7X KAPA | 30 μl | 252 μl | 536 μl |
| Ligation Buffer | | | |
| KAPA DNA Ligase | 10 μl | 84 μl | 179 μl |
| 30 uM Universal | 2.5 μl | 21.0 μl | 44.6 μl |
| Adaptor | | | |
| Total mastermix | 50 μl | 420 μl | 893 μl |
| volume | | | |

50 μl ligation mastermix was added to 60 μl of End repair& A-Tailing reaction product and incubated at room temperature for 15 minutes. The reaction was then subjected to an adaptor ligation cleanup reaction using Solid Phase Reversible Immobilization (SPRI) beads to reversibly bind DNA fragments to remove any remaining unbound adaptors and to concentrate DNA fragments in the library as in Table 9. SPRI beads were added to the ligation reaction product. The reaction mixture was incubated for 5 minutes at room temperature. Post incubation, the supernatant was removed and the reaction mixture was washed twice with 200 μl 80% EtOH. Beads were allowed to dry for 2-3 minutes. Beads were eluted in 50 μl elution buffer.

TABLE 9
Adaptor ligation cleanup reaction

| | |
|---|---|
| Adaptor ligation reaction product | 110 μl |
| SPRI beads | 88 μl |
| Total Volume per well/tube | 198 μl |

The barcoded library was then subjected to another amplification reaction as shown in Tables 10A-C.

TABLE 10A
Library amplification reaction mastermix

| Library Amplification | | 8 Libraries | 17 Libraries |
|---|---|---|---|
| Mastermix | 1 Library | (5% overage) | (5% overage) |
| 2X KAPA HiFi U + | 50 μl | 420 μl | 893 μl |
| Master Mix | | | |
| 10 uM PP695 Primer | 5.0 μl | 42 μl | 89 μl |

TABLE 10B
Library amplification reaction

| Library Amplification Reaction | |
|---|---|
| Library DNA | 40 μl |
| Library Amplification Mastermix | 55 μl |
| SI primer | 5 μl |
| Total Volume per well/tube | 100 μl |

TABLE 10C
Library amplification reaction conditions
Thermocycling

| Step | Temp | Duration | | Cycles |
|---|---|---|---|---|
| Initial Denaturation | 98° C. | 45 | sec | 1 |
| Denaturation | 98° C. | 20 | sec | repeat 11X |
| Annealing | 54° C. | 30 | sec | |
| Extension | 72° C. | 20 | sec | |
| Final Extension | 72° C. | 1 | min | 1 |

| | | | |
|---|---|---|---|
| Stop Reaction | 4° C. | Hold | Hold |

After the amplification reaction, the library was subjected to another clean up reaction using a PCR reaction clean up kit. The samples were then subjected to a sequencing reaction using an Illumina sequencer. In the test samples the mean duplication rate was found to be low which indicated a high library complexity as seen in the amplification rate (as high as 0.45). The unmapped fraction was found to be low (1%) and the ratio of reads in single cells was also found to be high.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-69. (canceled)

70. A method for processing a plurality of nucleic acid molecules, comprising:
(a) subjecting a nucleic acid molecule of said plurality of nucleic acid molecules to a nucleic acid amplification reaction, wherein said nucleic acid amplification reaction is performed in presence of at least one nucleotide analog, thereby generating an amplified nucleic acid molecule comprising said at least one nucleotide analog;
(b) contacting said amplified nucleic acid molecule with a modification dependent restriction enzyme;
(c) using said modification dependent restriction enzyme to subject said amplified nucleic acid molecule to fragmentation at a location at or in proximity to said at least one nucleotide analog to yield a plurality of nucleic acid fragments; and
(d) using a nucleic acid barcode molecule comprising a barcode sequence to barcode a nucleic acid fragment of said plurality of nucleic acid fragments, to yield a barcoded fragment comprising said barcode sequence.

71. The method of claim 70, wherein said nucleic acid amplification reaction is performed at a temperature between 25° C. and 35° C.

72. The method of claim 70, wherein said nucleic acid amplification reaction is performed using a high-fidelity polymerizing enzyme.

73. The method of claim 72, wherein said high-fidelity polymerizing enzyme is phi29 or a functional derivative thereof.

74. The method of claim 70, wherein any one or more of (a), (b), (c), and (d) is performed in a partition.

75. The method of claim 74, further comprising subjecting said barcoded fragment or derivative thereof to nucleic acid amplification.

76. The method of claim 75, wherein said nucleic acid amplification is polymerase chain reaction (PCR) or isothermal amplification.

77. The method of claim 70, wherein said nucleic acid molecule is from a single cell, from a cell bead or derived from a target nucleic acid molecule from said single cell or cell bead.

78. The method of claim 77, wherein in (a), said nucleic acid molecule or said target nucleic acid molecule is released from said single cell or cell bead.

79. The method of claim 70, wherein said at least one nucleotide analog is a methylated nucleotide.

80. The method of claim 70, wherein said modification dependent restriction enzyme is a methylation dependent restriction enzyme.

81. The method of claim 70, wherein said modification dependent restriction enzyme fragments said amplified nucleic acid molecule at said location at said least one nucleotide analog.

82. The method of claim 70, wherein said modification dependent restriction enzyme fragments said amplified nucleic acid molecule at a position up to 10 nucleotide bases from said at least one nucleotide analog.

83. The method of claim 70, wherein said fragmentation of said amplified nucleic acid molecule by said modification dependent restriction enzyme is performed at a temperature between 15° C. and 75° C.

84. The method of claim 70, wherein said nucleic acid barcode molecule is coupled to a bead.

85. The method of claim 84, further comprising, prior to (d), releasing said nucleic acid barcode molecule from said bead.

86. The method of claim 84, wherein said bead is a gel bead.

87. The method of claim 70, wherein said nucleic acid barcode molecule is from a plurality of nucleic acid barcode molecules, and wherein nucleic acid barcode molecules of said plurality of nucleic acid barcode molecules comprise a common barcode sequence and unique molecular identifier sequences.

88. The method of claim 70, further comprising subjecting said barcoded fragment or derivative thereof to nucleic acid sequencing to identify a sequence of at least a portion of said nucleic acid fragment and at least a portion of said barcode sequence.

89. The method of claim 74, wherein said partition is a droplet.

90. The method of claim 74, wherein said partition is a well.

91. The method of claim 70, wherein said nucleic acid fragment is barcoded by ligating said nucleic acid barcode molecule to said nucleic acid fragment.

92. The method of claim 91, wherein said ligating is performed at a temperature between about 16° C. and about 30° C.

93. The method of claim 70, wherein said nucleic acid fragment is barcoded using said nucleic acid barcode molecule as a primer and performing an extension reaction to extend said nucleic acid fragment.

94. The method of claim 70, wherein said modification dependent restriction enzyme is MspJI.
